(19) United States
(12) Patent Application Publication
(10) Pub. No.: US 2021/0322503 A1
(43) Pub. Date: Oct. 21, 2021

(54) HERBAL FORMULATION FOR CONSTIPATION AND GASTRIC TROUBLES

(71) Applicant: Aadil Shah, Ahmedabad (IN)

(72) Inventor: Aadil Shah, Ahmedabad (IN)

(21) Appl. No.: 17/147,469

(22) PCT Filed: Jul. 29, 2019
(86) PCT No.: PCT/IB2019/056459

§ 371 Date: Jan. 13, 2021

(30) Foreign Application Priority Data
Jul. 28, 2018 (IN) 201821028420

(51) Int. Cl. A61K 36/482; A61K 36/185; A61K 36/23; A61K 35/02; A61P 1/10

(52) U.S. Cl. CPC A61K 36/482; A61K 36/185; A61K 2236/15; A61K 35/02; A61P 1/10; A61K 36/23

(57) ABSTRACT

The present invention discloses a herbal formulation for curing mild to chronic constipation, acidity and gastric troubles. The formulation comprises of Cassia angutifolia, Terminalia chebula, Carum copticum, and Sanchal. Further, the formulation is prepared using grinding, pulverization and mixing. Further, the formulation can be administered orally in the form of a tablet, capsule, powder or liquid. Additionally, the herbal formulation helps promote proper digestion, by regulating bowel movements of the body.

### PRIORITY CLAIM

The present US national application claims the benefit and priority of international application PCT/IB2019/056459, filed Jul. 29, 2019, which, together with the present application, claim the benefit and priority of the earlier national application (India) 201821028420, filed July 28, 2018. Both of these earlier applications are incorporated herein as if restated in full.

### FIELD OF INVENTION

The present invention relates to the field of herbal formulation. Particularly, the invention relates to a herbal formulation and process of preparation thereof, which is effective in curing mild to chronic constipation, acidity and gastric troubles in humans. More particularly, the invention relates to a herbal formulation for constipation and gastric troubles.

### BACKGROUND OF INVENTION

Constipation is a condition in which a person has uncomfortable or infrequent bowel movements. Generally, a person is considered to be constipated when bowel movements result in passage of small amounts of hard, dry stool, usually fewer than three times a week.

People who are constipated may find it difficult and painful to have a bowel movement. Other symptoms of constipation include feeling bloated, uncomfortable, and sluggish. The hard and dry stools of constipation occur when the colon absorbs too much water or if the colon's muscle contractions arc slow or sluggish, causing the stool to move through the colon too slowly. Common causes of constipation are not enough fiber in the diet, not enough liquids, lack of exercise, medications, irritable bowel syndrome, changes in life or routine such as pregnancy or older age or travel, abuse of laxatives, ignoring the urge to have a bowel movement, specific diseases such as stroke, problems with the colon and rectum and problems with intestinal function such as chronic idiopathic constipation. Sometimes constipation can lead to complications such as hemorrhoids caused by straining to have a bowel movement or anal fissures caused when hard stool stretches the sphincter muscle or rectal prolapse caused by straining which leads to a small amount of intestinal lining to push out from the anal opening.

Constipation is a very complicated problem. Very few options are available from the synthetic drugs. Safe and effective drugs are available from herbal origin. Sonamukhi (Cassia Angustifolia) is useful in habitual costiveness. It lowers bowels, increases peristaltic movements of the colon by its local action upon the intestinal wall. It is used as expectorant, wound dresser, antidysentric, carminative and laxative. Useful in loss of appetite, hepatomegaly, spleenomegaly, indigestion, malaria, skin diseases, jaundice and anaemia.

U.S. Patents relating to Sonamukhi containing compositions include: U.S. Pat. No. 4,842,865, issued Jun. 27, 1989 to Durr et al. (relates to the use of glycofurol for liquidization of pharmaceutical preparations containing more than 50% phosphatidyl choline with a high content of unsaturated fatty acid, which preparations can contain another active ingredient, one of which is stated to be Sonamukhi extract); U.S. Pat. No. 4,595,592, issued Jun. 17, 1986 to Hietala (relates to a process for obtaining laxative compounds from Sonamukhi drug by extraction of Sonamukhi drug); U.S. Pat. No. 4,476,121, issued Oct. 9, 1984 to Moss (relates to orally administered compositions useful for treating constipation comprising a synergistic mixture of fruits, glycerine and compound Sonamukhi); U.S. Pat. No. 4,402,944, issued Sep. 6, 1983 to Callahan et al. (relates to polysulfonated sennoside A and B derivatives; Example describes an injectable oil comprising the active compound and sesame oil); and U.S. Pat. No. 4,256,875, issued Mar. 27, 1981 to Gabriel et al. (relates to a process for extraction of sennoside from Sonamukhi).

Harde (Terminalia chebula) has great reputation in Ayurvedic treaties for its curative effect over indigestion. According to Ayurvedic text, indigestion is the mother of all metabolic diseases. Harde has an ability to retrieve the normal digestion back and to maintain the balance of all three dosha. It is widely employed in Ayurvedic practice as a gentle laxative, blood purifier and digestive. Terminalia chebula Retz. (Combretaceae) (“TC”) has been extensively used in Ayurveda, Unani and Homoeopathic systems of medicines for improvement of different health conditions, e.g., constipation, diarrhoea, ulcers, gastroenteritis, asthma, cough, dyspnea, dyspepsia, hemorrhoids, candidiasis, parasites, malabsorption syndrome, hepatomegaly, vesicular and renal calculi, urinary discharges, tumors, skin diseases, leprosy, intermittent fever, rheumatism, arthritis, gout, neuropathy, paralysis, memory loss, epilepsy, depression, leucorrhea, diabetes, cardiovascular diseases, anorexia, and wounds, among others (See, B. Das, Materia Medica of Ayurveda (New Delhi: B. Jain Publishers, 1991), p. 8; K. R. Kirtikar and B. D. Basu, “Terminalia chebula.” In: Indian Medicinal Plants, (2nd Edn., Allahabad, India: Lolit Mohan Basu Publication, 1935), pp. 1020-23; and P. V. Sharma, Dravya Guna Vigyana (Vol. 2, Varanasi: Chaukhamba Bharati Academy, 1995), pp. 753-58). T. chebula fruit and its different solvent extractives were reported to exhibit hepatoprotective, cardioprotective, antimutagenic/anticarcinogenic, cytoprotective, radioprotective, antioxidant and adaptogenic, antimicrobial, antifungal, antiviral, antiamoebic, immunomodulatory, antidiabetic, wound healing, antispasmodic, and purgative activities in various animal models (S. S. Tasduq, et al., Human and Exp. Toxicol. (2006) 25: 11-18; H. Y. 5 Cheng, et al., Biol. Pharm.Bull. (2003) 26:1331-5; S. Kaur, et al.,

Mutagen Res. (1998) 419: 169-79; Suthienkul, et al., South-East Asian Journal Trop. Med., Public Health (1993) 24: 751-5; Ahmad, et al., J. Ethnopharmacol. (1998) 62: 183-93; and N. K. Rao, et al., BMC Complement Ahern. Med. (2006) 6: 127-32).

Ajwain (Carum copticum) has been used in the past for various therapeutic effects including bloating, fatigue, diarrhea, abdominal tumors, abdominal pain, respiratory distress, and loss of appetite. It has other health benefits such as antifungal, antioxidant, antibacterial, antiparasitic, and hypolipidemic effects.

Patent Publication No. WO2007057924A1 discloses a laxative composition comprising triphala as the bowel evacuant and lactulose as the sac charide component, optionally with at least one component selected from a group comprising peppermint, fennel, ajwain, mentha extract. The composition is useful as a pharmaceutical or a dietary supplement for the treatment diseases or disorders of the bowel primarily associated with constipation, diarrhoea or indigestion.

Patent Publication No. US20060115468 discloses a dietary supplement for treating and preventing gastrointestinal disorders. The composition comprises a blend of natural ingredients, including acidophilus, digestive enzymes and antioxidants. The other natural ingredients included in the composition are ginger root, fennel seed, peppermint, papain (from papaya), papaya fruit, bromelain (from pineapple), grapefruit pectin, apple pectin, carrot fiber, peach fiber, flaxseed, pysllium, oat bran fiber, prune powder, red clover, spikenard, spirulina, red beef extraction, Jerusalem artichoke, apple cider vinegar powder, fenugreek and Quinoa.

Constipation is often associated with several disorders such as gas, bloating, cramps, acidity, nausea, irritation, abdominal discomfort and pain. Although several prior art literature is available for the treatment of constipation but still there exists an unmet need for compositions which would be effective in the overall management of constipation including the prophylaxis or amelioration or treatment of not only the constipation but also associated disorders thus providing a better relief to the user.

Additionally, in spite of the above, the prior art generally does not reveal a herbal formulation which has the desired combination of safety, effectiveness and low cost which is needed for treating a large number of patients who have very different medical conditions and socio-economic backgrounds.

Accordingly, there is a continuous search in the art for a medicine or herbal formulation that is safe, useful and inexpensive for the prevention and treatment of constipation and gastric troubles. The novel compositions of the present invention overcomes such limitations of prior art.

### SUMMARY OF INVENTION

The present invention discloses a herbal formulation for curing mild to chronic constipation and gastric troubles. The formulation comprises of Cassia angutifolia, Terminalia chebula, Carum copticum, and Sanchal. Further, the herbal formulation helps promote proper digestion, by regulating bowel movements of the body.

In one aspect of the invention discloses the process of preparation of the herbal formulation. The raw ingredients Cassia angutifolia, Terminalia chebula, Carum copticum, and Sanchal in original dry herbal form are procured, got together and mixed (in respective ratios) for further process of grinding through pulverization Further, the mixture is grinded through pulveriser wherein the mixed formulation (i.e the dry herbal raw ingredients mixed together in respective ratios) is then put through the pulveriser for grinding. The griding process leads to a loss of ˜10% of the weighted formulation in this step. Furthermore, the grinded mixture is processed by sifting, wherein the matter is passed through the sift for gaining even more fine powder, where there might be a loss of ˜1% of the net weight and it removes any granules or larger particles. Moreover, post sifting the entire mixture is even further mixed, to ensure thorough mixing of the ingredients and to gain uniformity.

In another aspect of the invention discloses the process of preparation of capsule of the herbal formulation. Wherein the Ingredients in the form of dry herbal extracts are procured. The dry extract is made via a standard extraction process using a “Clavenger Apparatus” are process; then a quath (“boiled syrup formulation”) is formed; the quath is dried and converted to dry extract which is a concentrated dry fine powder of the herb. Further, the dry extracts of the ingredients are then mixed as per respective ratios, the mixed formulated ingredients are then undergo sifting to remove larger particles and achieve more fineness, which also removes any contaminants. The mixture is then further mixed to achieve uniformity and then excipients are added in respective ratios (for better adsorption within the body). Moreover, the final mixture is then filled in the capsules through standard encapsulation process using capsules of either gelatin or veg. (rice) based.

In general, in another aspect of the invention, the herbal formulation helps in mild to chronic constipation and gastric troubles. Further the herbal formulation helps promote proper digestion, by regulating bowel movements of the body. Furthermore, the herbal formulation to be taken consecutively upto the dosage prescribed for a week to obtain best results.

In another aspect of the present invention, the in case of chronic or severe conditions, the dosage may be increased for the first week to obtain best results.

In yet another aspect of the present invention, the herbal formulation for treatment of acute and chronic constipation, acidity and gastric troubles is disclosed. The formulation comprises of Sonamukhi, Harde, Ajwain, Sanchal and pharmaceutically acceptable excipient. The formulation can be administered orally in the form of a tablet, capsule, powder or liquid.

### DETAILED DESCRIPTION OF INVENTION

The following description is full and informative description of the best method presently contemplated for carrying out the present invention which is known to the inventors at the time of filing the patent application. Of course, many modifications and adaptations will be apparent to those skilled in the relevant arts in view of the following description in view of the accompanying drawings and the appended claims. While the system and method described herein are provided with a certain degree of specificity, the present technique may be implemented with either greater or lesser specificity, depending on the needs of the user. Further, some of the features of the present technique may be used to advantage without the corresponding use of other features described in the following paragraphs. As such, the present description should be considered as merely illustrative of the principles of the present technique and not in limitation thereof, since the present technique is defined solely by the claims.

The present invention relates to the field of herbal formulation. Particularly, the invention relates to a herbal formulation and process of preparation thereof, which are effective in curing mild to chronic constipation and gastric troubles in humans. More particularly, the invention relates to a herbal formulation for constipation and gastric troubles.

In one embodiment of the invention a herbal formulation for curing mild to chronic constipation and gastric troubles is disclosed. The formulation comprises of Cassia angutifolia, Terminalia chebula, Carum copticum, and Sanchal. Further, the herbal formulation helps promote proper digestion, by regulating bowel movements of the body.

In another embodiment of the invention Sonamukhi (Cassia Angustifolia) is useful in habitual costiveness, which lowers bowels, increases peristaltic movements of the colon by its local action upon the intestinal wall. Further, Sonamukhi is useful in loss of appetite, hepatomegaly, spleenomegaly, indigestion, malaria, skin diseases, jaundice and anaemia. Furthermore, Harde (Terminalia chebula) has an ability to retrieve the normal digestion back and to maintain the balance of all three dosha. Harde is widely employed in Ayurvedic practice as a gentle laxative, blood purifier and digestive. Moreover, Ajwain (Carumcopticum) is used for various therapeutic effects including bloating, fatigue, diarrhea, abdominal tumors, abdominal pain, respiratory distress, and loss of appetite. It has other health benefits such as antifungal, antioxidant, antibacterial, antiparasitic, and hypolipidemic effects. Additionally, Sanchal (Sauvarchala lavana) is a type of salt and constituted mainly with Sodium chloride with Iron and sulphides. Sanchal is used in the formulation of medicines for abdominal tumors, bloating, abdominal colic, constipation, heart, throat, belching. Sanchal is also used in the preparation of medicines for asthma, eczema, liver and spleen disorders, urinary disorders, digestion and metabolism, fever, menorrhagia, leucorrhoea.

In another embodiment of the invention the herbal formulation prepared through disclosed process having Cassia angutifolia, Terminalia chebula, Carum copticum, and Sanchal in pure form provides effective treatment to mild to chronic constipation, acidity and gastric troubles. The effectiveness of the formulation depends on the ratio of the ingredients in the mixture, as the disclosed ingredients are having therapeutical effect, while the combination of all ingredients, is having superior efficacy compared to individual ingredients. Furthermore, there are plenty of other herbs having medicinal effect in relation to ayurvedic treatment, while the herbal formulation of Cassia angutifolia, Terminalia chebula, Carum copticum, and Sanchal is having superior therapeutic effect in treating the mild to chronic constipation, acidity and gastric troubles.

In another embodiment of the invention the process of preparation of the herbal formulation is disclosed. The raw ingredients Cassia angutifolia, Terminalia chebula, Carum copticum, and Sanchal in original dry herbal form are procured, got together and mixed (in respective ratios) for further process of grinding through pulverization Further, the mixture is grinded through pulveriser wherein the mixed formulation (i.e the dry herbal raw ingredients mixed together in respective ratios) is then put through the pulveriser for grinding. The griding process leads to a loss of ˜10% of the weighted formulation in this step. Further, the grinded mixture is processed by sifting, wherein the matter is passed through the sift for gaining even more fine powder, where there might be a loss of ˜1% of the net weight and it removes any granules or larger particles. Moreover, post sifting the entire mixture is even further mixed, to ensure thorough mixing of the ingredients and to gain uniformity.

In another embodiment of the invention the process of preparation of capsule of the herbal formulation is disclosed. Wherein the Ingredients in the form of dry herbal extracts are procured. The dry extract is made via a standard extraction process using a “Clavenger Apparatus” are process; then a quath (“boiled syrup formulation”) is formed; the quath is dried and converted to dry extract which is a concentrated dry fine powder of the herb. Further, the dry extracts of the ingredients are then mixed as per respective ratios, the mixed formulated ingredients are then undergo sifting to remove larger particles and achieve more fineness, which also removes any contaminants. The mixture is then further mixed to achieve uniformity and then excipients are added in respective ratios (for better adsorption within the body). Moreover, the final mixture is then filled in the capsules through standard encapsulation process using capsules of either gelatin or veg. (rice) based.

In general, in another embodiment of the invention, the herbal formulation helps in mild to chronic constipation and gastric troubles. Further the herbal formulation helps promote proper digestion, by regulating bowel movements of the body. Furthermore, the herbal formulation to be taken consecutively upto the dosage prescribed for a week to obtain best results.

In another aspect of the present invention, in the case of chronic or severe conditions, the dosage may be increased for the first week to obtain best results.

In yet another aspect of the present invention, the herbal formulation for treatment of acute and chronic constipation and gastric troubles is disclosed. The formulation comprises of Sonamukhi, Harde, Ajwain, Sanchal and pharmaceutically acceptable excipient. The formulation can be administered orally in the form of a tablet, capsule, powder or liquid.

In an exemplary embodiment of the invention the formulation comprises of Cassia angutifolia, Terminalia chebula, Carum copticum, and Sanchal in the ratio of 2:1:1:1. The exemplary formulation is mild and required to be taken higher dosage for first week for effective treatment of ailment.

In another exemplary embodiment of the invention the formulation comprises of Cassia angutifolia, Terminalia chebula, Carum copticum, and Sanchal in the ratio of 6:2:1:1, which is required to be taken in lower dosage for effective treatment. However, the amount of formulation needs to be decided based on the physiological condition along with severity of the ailment

In still another embodiment of the invention, preparation of the herbal formulation for curing mild to chronic constipation, acidity and gastric troubles, the preparation comprises steps of : mixing Cassia angutifolia, Terminalia chebula, Carum copticum, and Sanchal; grinding the mixed formulation through pulverization; sifting the grinded formulation for gaining more fine powder; and further mixing the grinded formulation to obtain a uniform herbal formulation.

In still another aspect of the invention, the invention provides following advantages:
The formulation can be used for controlling, treating or bringing back to normalcy in constipation as a mild to severe condition.
The formulation helps in controlling and treating acidity in all conditions including chronic acidity.
The formulation helps in providing relief in the gastric troubles.

1 The formulation can also be used as catalyst to increase appetite in conditions where loss of appetite is caused by constipation.
The formulation can be used to prevent, control or treat any conditions arising out of constipation acidity or gastric troubles by using the herbal formulation prepared with different ratio of ingredients.

### Evaluation of the Formulation of the Present Invention:

A. Procedure:

Fifty (50) patients suffering from chronic constipation were administered 500 mg formulation of the present invention comprising comprises of Cassia angutifolia, Terminalia chebula, Carum copticum, and Sanchal in the ratio of 2:1:1:1. The patients were administered the said dose once per day for one week.

### Observation:

TABLE 1
Effect of the formulation

| S. No. | No. of Patients | Effect |
|---|---|---|
| 1. | 43 | A |
| 2. | 7 | B |
| 3. | 0 | C |

A: Complete relief;
B: Relief but not completely;
C: No effect

### Observation and Results:

The formulation of the present invention has effectively treated 86% of the patients; whereas remaining 14% patients also felt relief after, and none of the patients reported no effect. the administration of the formulation of the present invention. It is inferred that all the patients were administered a standard dose of 500 mg; irrespective of their longevity of their gastric trouble i.e. chronic constipation. To get 100% results a precise dose optimization is required based on age, weight, history of gastric trouble and other parameters.

B. Procedure:

Fifty (50) patients suffering from chronic constipation were administered two teaspoon (˜8 gm) formulation of the present invention comprising comprises of Cassia angutifolia, Terminalia chebula, Carum copticum, and Sanchal in the ratio of 6:2:1:1. The patients were administered the said dose once per day for one week.

TABLE 2
Effect of the formulation

| S. No. | No. of Patients | Effect |
|---|---|---|
| 1. | 44 | A |
| 2. | 6 | B |
| 3. | 0 | C |

A: Complete relief;
B: Relief but not completely;
C: No effect

### Observation and Results:

The formulation of the present invention has effectively treated 88% of the patients; whereas remaining 12% patients also felt relief after, and none of the patients reported no effect. The administration of the formulation of the present invention. It is inferred that all the patients were administered a standard dose of two teaspoon (˜8 g); irrespective of their longevity of their gastric trouble i.e. chronic constipation. To get 100% results a precise dose optimization is required based on age, weight, history of gastric trouble and other parameters.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

While, the following description is presented to enable a person of ordinary skill in the art to make and use the invention and is provided in the context of the requirement for obtaining a patent. The present description is the best presently-contemplated method for carrying out the present invention. Various modifications to the preferred embodiment will be readily apparent to those skilled in the art and the generic principles of the present invention may be applied to other embodiments, and some features of the present invention may be used without the corresponding use of other features. Accordingly, the present invention is not intended to be limited to the embodiment shown but is to be accorded the widest cope consistent with the principles and features described herein.

Many modifications of the present invention will be apparent to those skilled in the arts to which the present invention applies. Further, it may be desirable to use some of the features of the present invention without the corresponding use of other features.

Accordingly, the foregoing description of the present invention should be considered as merely illustrative of the principles of the present invention and not in limitation thereof.

What is claimed is:

1. A herbal formulation for curing mild to chronic constipation, acidity, and gastric troubles, the formulation comprising Cassia angutifolia, Terminalia chebula, Carum copticum, and Sanchal.

2. The herbal formulation as claimed in claim 1, wherein the formulation comprises Cassia angutifolia, Terminalia chebula, Carum copticum, and Sanchal in a ratio of 2:1:1:1.

3. The herbal formulation as claimed in claim 1, wherein the formulation comprises Cassia angutifolia, Terminalia chebula, Carum copticum, and Sanchal in a ratio of 6:2:1:1

4. The herbal formulation as claimed in claim 1, wherein the formulation is a powder or capsule and comprises pharmaceutical excipients.

5. A method of preparation of herbal formulation for curing mild to chronic constipation, acidity, and gastric troubles, the preparation comprising the steps of :
mixing Cassia angutifolia, Terminalia chebula, Carum copticum, and Sanchal; grinding the mixed formulation through pulverization;
sifting the grinded formulation to remove granules; and
further mixing the grinded formulation to obtain a uniform herbal formulation.

6. The method as claimed in claim 5, wherein the preparation comprises Cassia angutifolia, Terminalia chebula, Carum copticum, and Sanchal in a ratio of 2:1:1:1.

7. The method as claimed in claim 5, wherein the preparation comprises Cassia angutifolia, Terminalia chebula, Carum copticum, and Sanchal are in a ratio of 6:2:1:1.

8. The herbal formulation as claimed in claim 1, wherein the formulation is prepared in a powder or capsule and comprises pharmaceutical excipients.
